(19) United States
(12) Patent Application Publication
(10) Pub. No.: US 2002/0025967 A1
(43) Pub. Date: Feb. 28, 2002

(54) N-alkylpiperdine analogs and uses thereof in treating addictions

(76) Inventor: Miles P. Smith, Belmont, MA (US)

Correspondence Address: Patent Group, Foley, Hoag & Eliot LLP, One Post Office Square, Boston, MA, 02109-2170, US

(21) Appl. No.: 09/739,184

(22) Filed: Dec. 18, 2000

Related U.S. Application Data
(60) Provisional application No. 60/171,139, filed on Dec. 16, 1999.

(51) Int. Cl. A61K 31/517; A61K 31/454; C 07D 4 1/02; A61K 31/445

(52) U.S. Cl. 514/259; 514/317; 514/323; 544/284; 546/200; 546/225

(57) ABSTRACT

One aspect of the present invention relates to alkylpiperdine compounds and pharmaceutical compositions thereof. A second aspect of the present invention relates to the use of alkylpiperdine compounds and pharmaceutical compositions thereof as promoters of 5-HT_{2A} antagonistic activity. A third aspect of the invention relates to methods of treating addiction using a compound of the invention or a pharmaceutical compositions thereof.

### BACKGROUND OF INVENTION

Cocaine use in the US has reached endemic levels with 1.5 million current cocaine users in 1997, according to estimates by 1997 National Household Survey on Drug Abuse (NHSDA). Due to the high incidence of cocaine dependence and the great profits arising through the cocaine distribution network, it has been estimated that drug associated crime alone costs $50 billion/year. Combining this with other economic costs including health care increase the monetary cost to over $100 billion annually. While the magnitude of these numbers is great, the total cost including the incalculable costs due to human suffering such as domestic violence, reduced productivity and lost opportunities undoubtedly significantly increase the cost of cocaine abuse to society. While the number of occasional users had sharply declined over the previous 10 years, the number of frequent users remained relatively constant, and the amount of cocaine consumed has remained steady. Taken together these findings suggest that there are increasing numbers of addicted individuals that require immediate therapies. As cocaine's pharmacological properties stem largely from its ability to inhibit the dopamine transporter (DAT), many intervention strategies have focused on dopaminergic agents. (Smith, M. P.; Hoepping, A.; Johnson, K.

M.; Trzcinska, M.; Kozikowski, A. P. Dopaminergic agents for the treatment of cocaine abuse Drug Discov Today 1999, (4) 322-332, Caroll, F. I.; Howell, L. L.; Kuhar, M. J.

Pharmacotherapies for treatment of Drug Abuse: Preclinical aspects. J Med. Chem. 1999, 42.) Other strategies have also been explored utilizing agents that interact with other sites including the serotonergic or opiate systems. While success has been noted in preliminary trials for several therapies, no medication has exhibited efficacy in double blind clinical trials.(Nathan, K. I.; Bresnick, W. H.; Batki, S. L. Cocaine abuse and dependance: Approaches to management. CNS Drugs 1998, 10, 43-59. Grabowski, J.; Rhoades, H.; Elk, R.; Schmitz, J.; Davis, C.; Creson, D.; Kirby, K. Fluoxetine is ineffective for treatment of cocaine dependence or concurrent opiate and cocaine dependence: two placebo-controlled double-blind trials. J Clin. Psychopharmacol. 1995, 15, 163-174).

Several studies have investigated treatments during initial withdrawal from cocaine using a strategy of pharmacological antagonism of cocaine's dopaminergic properties (.Dewey, S. L.; Morgan, A. E.; Ashby, C. R.; Horan, B.; Kushner, S. A.; Logan, J.; Volkow, N. D.; Fowler, J. S.; Gardner, E. L.; Brodie, J. D. A novel strategy for the treatment of cocaine abuse Synapse 1998, 30, 99-111.

In addition, there is emerging evidence that monoaminergic properties are likely to be required in potential pharmacotherapies. First, it is anticipated that DA (dopamine) reuptake inhibition is needed to alleviate the anhedonia that is associated with the transient decreases in dopaminergic neurotransmission following cessation of cocaine use.(Little, K. Y.; Patel, U. N.; Clark, T. B.; Butts, J. D. Alteration of brain dopamine and serotonin levels in cocaine users: a preliminary report. Am. J Psychiatry 1996, 153, 1216-1218) Additional inhibitory activity at the serotonin transporter (5-HTT) may serve to counteract the increase in craving associated with the administration of a DA reuptake inhibitor. This strategy is supported by the reported success of a combination of the 5-HT releaser fenfluramine with the DA releaser phentermine or pemoline in pilot studies for cocaine addiction treatment. (Rothman, R. B.; Gendron, T. M.; Hitzig, P. Combination use of fenfluramine and phentermine in treatment of cocaine addiction: A pilot case series J Subst. Abuse Treat. 1994, 11, 273-275. Rothman, R. B.; Gendron, T. M.; Hitzig, P. Treatment of Alcohol and Cocaine Addiction by the combination of pemoline and fenfluramine: A pilot case series J Subst. Abuse Treat. 1995, 12, 449-453.)

Studies have reported significant correlation between regional brain metabolism in the orbitofrontal and prefrontal cortices and cocaine craving in abstinent patients. Recent studies have shown increases in metabolism in these area following treatment with the DAT inhibitor methylphenidate, resulted in an increase in craving in human cocaine addicts.(Volkow, N. D.; Wang, G. F.; Fowler, J. S.; Hitzemann, R.; Angrist, B.; Gatley, S. J.; Logan, J.; Ding, Y. S.; Pappas, N. Association of methylphenidate-induced craving with changes in right striato-orbito frontal metabolism in cocaine abusers: implications in addiction. Am. J Psychiatry 1999, 156,19-26) These suggest that the possible use of agents that are selective agonists of cocaine at the DAT may not be effective for the treatment of cocaine withdrawal. Such agents may help to relieve the anhedonia resulting from the putative DA deficits, but also serve as the introceptive cue that enhances craving. Serotonergic systems have been most closely implicated in craving. This link is primarily related to the correlation of the compulsive cocaine seeking behavior (craving) as a form of obsessive-compulsive disorder (OCD). This is further supported by the observation that patients with OCD, exhibit abnormal metabolism in the orbitofrontal cortex, and treatment with a SSRI serves to ameliorate the effects of this disorder. Previous clinical trials would suggest that the use of a pure SSRI would not likely result in significant efficacy for the treatment of cocaine withdrawal.(Rothman, R. B.; Glowa, J. R. A review of the effects of doparninergic agents on humans, animals and drug-seeking behavior, and its implications for medications development. Mol. Neurobio. 1995, 11, 149. Brody, A. L.; Saxena, S.; Schwartz, J. M.; Stoessel, P. W.; Maidment, K.; Phelps, M. E.; Baxter, L. R. Jr. FDG-PET predictors of response to behavioral therapy and pharmacotherapy in obsessive compulsive disorder Psychiatry Res. 1998, 84, 1-6. Batki, S. L.; Washburn, A. M.; Delucchi, K.; Jones, R. T. A controlled trial of fluoxetine in crack cocaine dependence. Drug Alcohol Depend. 1996, 41, 13 7-142.)

As noted above the addictive properties of cocaine are modulated by serotinergic Systems. This modulation has been postulated to involve the activation of the 5-HT_{2A} receptors. The 5-HT₂ antagonists ketanserin and ritanserin do not substitute for cocaine in drug discrimination studies in non-human primates, but antagonize the discriminative effects of cocaine. 5-HT₂ antagonists have also been reported to antagonize cocaine-induced convulsions and cocaine-induced elevations in locomotor activity. This cocaine antagonist activity may be due to the antagonism of the 5-HT modulation of DA. O'Neill, M. F.; Heron-Maxwell, C. L.; Shaw, G. 5-HT₂ receptor antagonism reduces hyperactivity induced by amphetamine, cocaine, and MK-801 but not D1 agonist C-APB Pharmacol. Biochem. Behav. 1999, 63, 23 7-243 Schama, K. F.; Howell, L. L.; Byrd, L. D. Serotinergic modulation of the discriminative stimulus effects of cocaine in squirrel monkeys. Psychopharmacol. 1997, 132, 27-34 Ritz, M. C.; George, F. R. Cocaine-induced convulsions: pharmacological antagonism at serotonergic, muscarinic and sigma receptors. Psychopharmacol. 1997, 129, 299-3 10. Kelland, M. D.; Freeman, A. S.; Chiodo, L. A. Serotonergic afferent regulation of the basic physiology and pharmacological responsiveness of migrostriatal dopamine neurons. J Pharmacol. Exp. Ther. 1990, 253, 803-811.)

Despite the apparent contradictions in the interaction of 5-HT_{2A} antagonists with cocaine there are several lines of evidence from animal models that suggest that 5-HT_{2A} antagonists may be useful in treating various forms of drug abuse including cocaine abuse. In rats, ritanserin reduced the preference and consumption of drugs of abuse including cocaine in a free choice, drinking paradigm. No decrease in sucrose preference was noted during this study. Ritanserin also attenuated the sleep-wakefulness alterations noted in rats during withdrawal from cocaine. Based on these observations and the known improvement in mood and drive in depressed humans resulting from treatment with ritanserin it has been evaluated as a potential treatment to reduce cocaine consumption and craving in double-blind clinical trials. Feighner, J. P. Mechanism of action of antidepressant medications. J Clin. Psychiatry 1999, 60 S4,4-11 Pertz, H. H.; Milhahn, H.; Eich, E. Cycloalkanecarboxylic esters derived from lysergol, dihydrolysergol-I, and elymoclavine as partial agonists and antagonists at rat 5-HT_{2A} receptors: pharmacological evidence that the indolo[4,3-fg]quinoline system of the ergolines is responsible for high 5-HT_{2A} receptor affinity. J Med. Chem. 1999, 42, 659-668).

This study reported small, although not statistically significant reduction in blood benzoylecognine levels for the ritanserin treatment group. Such activity of the 5-HT₂ antagonist, useful to mask some of the behavioral effects of cocaine such as its locomotor stimulation, and therefore be a useful adjunct in a polypharmacophoric approach to cocaine treatment. Additional benefit may be derived as a direct consequence of the antidepressant properties of 5-HT_{2A} antagonists. In this direction single molecular entities were designed that exhibit significant activity at the 5-HT_{2A} receptor as well as potent inhibition of monoamine reuptake.

Several phases have been postulated in the withdrawal from cocaine dependence, although some dispute remains as to the existence of distinct phases, a period of anhedonia and high craving following cessation of cocaine use is generally accepted. Behavioral observations have identified a window of the first 10 weeks of abstinence in which susceptibility to relapse is the greatest. To increase the success rate for outpatient treatment programs, there is a need to develop pharmaceutical agents that are capable of assisting patient management during this period. (Brower, K. J.; Paredes, A. Cocaine withdrawal. Arch. Gen. Psychiatry 1987, 44, 297-298. Lago, J. A.; Kosten, T. R. Stimulant withdrawal. Addiction 1994, 89, 1477-1481 Fischman, M. W.; Schuster, C. R. (1982) Cocaine self-administration in humans. Fed. Proceed. 41, 241).

A treatment that is capable of ameliorating some of the symptoms of withdrawal including anhedonia and craving in addition to antagonizing some of the effects of cocaine during recidivism should result in a dramatic improvement in abstinence. Effectively, a pharmacological agent able to treat anhedonia and craving (pharmacological agonist) during the initial stages of abstinence and to prevent some of the behavioral effects of cocaine (behavioral antagonism) in the event of recidivism would provide an effective treatment stratagem.

### SUMMARY OF THE INVENTION

In one aspect, the present invention provides a compound of formula (I)

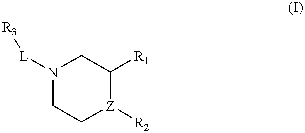

Where Z is NR₆, —C(R₄)(R₅)—, or —O—;

L is a (C1-C6)alkyl or (C1-C6)alkoxy, wherein any alkyl may be optionally substituted with 1, 2 or 3 substituents independently selected from halo, nitro, cyano, hydroxy, ketone, (C1-C6)alkoxy;

R₁ is —C(═O)ORₐ, cyano, (C1-C6)alkyl, (C1-C6)alkanoyl, (C2-C6)alkenyl, (C2-C6)alkynyl;

R₂ is (C6-C10)aryl, 5-10 membered heteroaryl, (C6-C10)aryl(C1-C6)alkyl, (C1-C6)alkyl(C6-C10)aryl, 5-10 membered heteroaryl(C1-C6)alkyl, (C6-C10)arylcarbonyl, biphenyl, or 5-10 membered heteroarylcarbonyl, wherein any aryl, biphenyl, or heteroaryl substituent may optionally be substituted on carbon with 1, 2, 3 substituents independently selected from the group consisting of halo, nitro, cyano, hydroxy, (C1-C6)alkyl, (C2-C6)alkenyl, (C2-C6)alknyl, (C1-C6)alkoxy, (C2-C6)acyloxy, trifluoromethyl;

R₃ is a (C6-C10)aryl, 5-10 membered heteroaryl, (C6-C10)aryl(C1-C6)alkyl, (C1-C6)alkyl(C6-C10)aryl, 5-10 membered heteroaryl(C1-C6)alkyl, (C6-C10)arylcarbonyl, biphenyl, heterocyclyls, or 5-10 membered heteroarylcarbonyl, wherein any aryl, biphenyl, or heteroaryl substituent may optionally be substituted on carbon with 1, 2, 3 substituents independently selected from the group consisting of halo, nitro, cyano, hydroxy, (C1-C6)alkyl, (C2-C6)alkenyl, (C2-C6)alknyl, (C1-C6)alkoxy, (C2-C6)acyloxy, trifluoromethyl;

R₄ and R₅ are independently hydrogen or (C1-C6)alkyl;

R₆ is a halogen, (C1-C6)alkyl, (C1-C6)alkanoyl, (C2-C6)alkenyl, (C2-C6)alkynyl, triflouromethyl, aryl(C1-C4)alkyl, heteroaryl(C1-C4)alkyl, aryl(C1-C4)alkanoyl, or heteroaryl(C1-C4)alkanoyl;

Rₐ is hydrogen, (C1-C4) alkyl, aryl, heteroaryl, aryl(C1-C4)alkyl, or heteroaryl(C1-C4)alkyl.

In another aspect, the present invention provides a formulation, comprising a compound of the present invention and a pharmaceutically acceptable excipient.

In certain embodiments, the present invention provides a method of promoting 5-HT_{2A} antagonistic activity , in a patient, comprising the step of administering to a patient in need of 5-HT_{2A} antagonistic activity a therapeutically effective amount of a compound or formulation of the present invention.

In certain embodiments, the present invention provides a method of promoting inhibitory activity at the dopamine (DAT) and/or serotonin (5-HTT) and/or norepinephrine (NET) receptors in a patient, comprising the step of administering to a patient in need of inhibitory activity a therapeutically effective amount of a compound or formulation of the present invention.

In certain embodiments, the present invention provides a method of simultaneously promoting 5-HT_{2A} antagonistic activity and promoting inhibitory activity at the dopamine (DAT) and/or serotonin (5-HTT) and/or norepinephrine (NET) receptors in a patient, comprising the step of administering to a patient in need of 5-HT_{2A} antagonistic activity and inhibitory activity at the dopamine (DAT) and/or serotonin (5-HTT) and/or norepinephrine SET) receptors a therapeutically effective amount of a compound or formulation of the present invention.

In certain embodiments, the present invention provides a method of treating addiction to addictive substances in a patient, comprising the step of administering to a patient having an addiction a therapeutically effective amount of a compound or formulation of the present invention.

In certain embodiments, the present invention provides a method of treating cocaine addiction in a patient, comprising the step of administering to a patient having cocaine addiction a therapeutically effective amount of a compound or formulation of the present invention.

### BRIEF DESCRIPTION OF THE DRAWINGS

FIG. 1. Illustrates the synthesis of compounds that can be used to prepare compounds of formula I.

FIG. 2. Illustrates the biological activity of compounds that can be used to prepare compounds of formula I.

FIG. 3. Synthetic strategy for the compounds of formula I.

FIG. 4. Illustrates the structure and features of compounds that can be used to prepare compounds of formula I.

FIG. 5. Illustrates the synthesis of compounds 6 and 8.

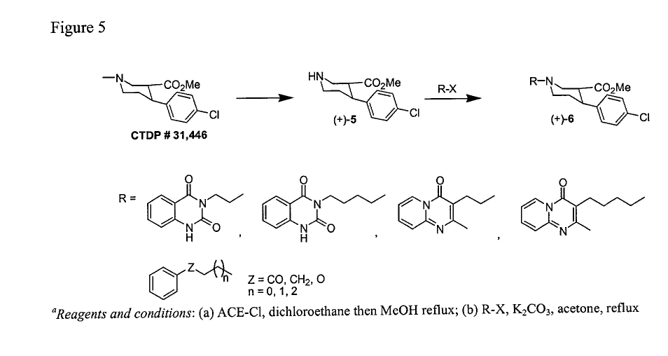

FIG. 6. Illustrates the synthesis of enantiomers of 6 and 8.

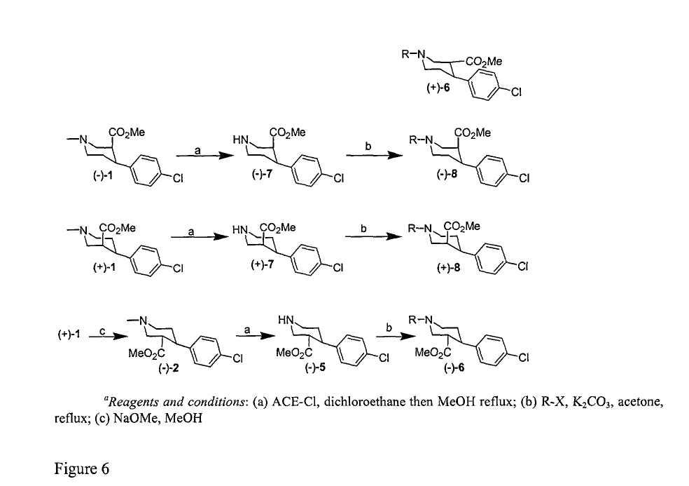

FIG. 7. Illustrates the synthesis of compounds 9, 10, 11, 12, 13.

### DETAILED DESCRIPTION OF THE INVENTION

Definitions

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylberzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, azetidine, azepine, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle”, as used herein, refers to an aromatic or non-aromatic ring in which each atom of the ring is carbon.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines, e.g., a moiety that can be represented by the general formula:

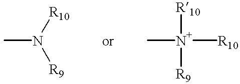

wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “acylamino” is art-recognized and refers to a moiety that can be represented by the general formula:

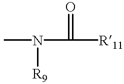

wherein R₉ is as defined above, and R′₁₁ represents a hydrogen, an alkyl, an alkenyl or —(CH₂)ₘ—R₈, where m and R₈ are as defined above.

The term “amido” is art recognized as an amino-substituted carbonyl and includes a moiety that can be represented by the general formula:

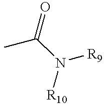

wherein R₉, R₁₀ are as defined above. Preferred embodiments of the amide will not include imides which may be unstable.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ—R₈, wherein m and R₈ are defined above. Representative alkylthio groups include methylthio, ethyl thio, and the like.

The term “carbonyl” is art recognized and includes such moieties as can be represented by the general formula:

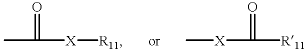

wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiolcarbonyl” group. Where X is a sulfur and R₁₁ or R′₁₁ is not hydrogen, the formula represents a “thiolester.” Where X is a sulfur and R₁₁ is hydrogen, the formula represents a “thiolcarboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₈, where m and R₈ are described above.

The term “sulfonate” is art recognized and includes a moiety that can be represented by the general formula:

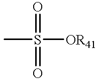

in which R₄₁ is an electron pair, hydrogen, alkyl, cycloalkyl, or aryl.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, it may be isolated using chiral chromatography methods, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g., exhibit significant inhibiting activity at DAT, HTT, and NET, and exhibit 5-HT_{2A} antagonist activity), wherein one or more simple variations of substituents are made which do not adversely affect the efficacy of these compounds to exhibit these properties. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

General

The instant invention features a series of compounds that exhibit significant activity at the 5-HT_{2A} receptor as well as potent inhibition of monoamine reuptake. Thus, the invention provides therapeutic agents and compounds for the treatment of cocaine abuse.

Processes and intermediates useful for preparing compounds of formula (I) are provided as further embodiments of the invention and illustrated by the following procedures.

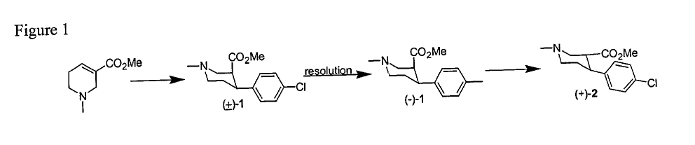

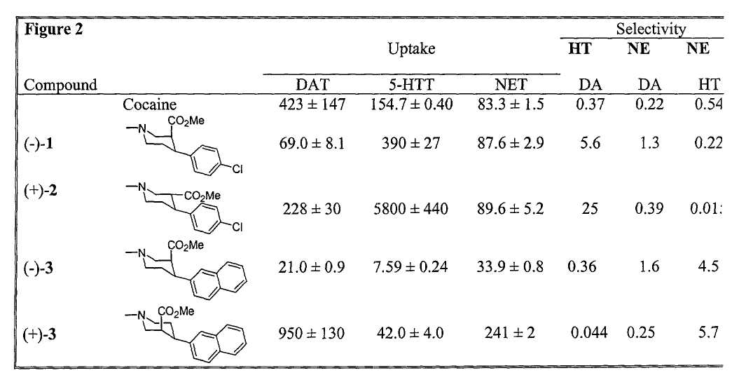

FIG. 1 delineates the chemistry that has been previously developed to gain access to intermediates of this series of compounds. These analogs arise from the addition of an aryl Grignard reagent to the free base of arecoline. The mixture of cis and trans disubstituted piperidines obtained are separated by chromatography/crystallization to afford the pure cis analog. The racemic material obtained is readily resolved by co-crystallization with (+) or (−) dibenzoyl tartaric acid to afford enantiomerically pure (−)-cis analog [(−)-1] or the corresponding (+)-cis enantiomer. The absolute configuration of these analogs has been confirmed by crystallography of the dibenzoyl tartaric acid salt. The optically pure cis enantiomers can be converted to their respective trans isomers, such as ((+)-2) using catalytic NaOMe in MeOH. This route allows for the introduction of a wide number of variously substituted aryl groups as well as the facile preparation of both enantiomers. The versatility of this route has been exploited to prepare a library of over 75 potent monoamine reuptake inhibitors with a range of selectivities. This library has been examined for in vitro ability to inhibit the high affinity uptake of DA, 5-HT and NE using synaptosomes prepared from rat striatum, midbrain, and cortex, respectively. As a result of this, structure activity relationships for this library is available to rationally design molecules with desired monoamine selectivity. The uptake data for this intermediate set of compounds is expressed as a Kᵢ and the selectivity as a ratio the Kᵢ values for selected compounds is shown in FIG. 2.

Behavorial properties of [(+)-2] include partial substitution in cocaine or amphetamine drug discrimination assays, antagonism of cocaine-induced convulsions, inhibition of cocaine-induced hyperlocomotion (20 mg/kg cocaine, AD₅₀=21 mg/kg), and antagonism of the cocaine induced reductions in brain-stimulation reward (BSR) thresholds. (Kozikowski, A. P.; Araldi, G. L.; Boja, J.; Meil, W. M.; Johnson, K M.; Flippen-Anderson, J. L.; George, C.; Saiah, E. Chemistry and pharmacology of the piperidine-based analogues of cocaine. Identification of potent DAT inhibitors lacking the tropane skeleton. J. Med. Chem. 1998, 41, 1962-1969, Mitkus, R. J.; Katz, J. L. Study report for CTDP 31,446. Psychobiology section, NIDA Intramural Research Program.) Based on its novel properties [(+)-2] it was recently found that this compound exhibited 5-HT_{2A} affinity (K₁=778 nM) in the National Institute of Mental Health's Psychoactive Drug Screening Program. From this screening and additional screenings through commercial laboratories (MSD PanLab's SpectrumScreen™) no significant activity (no activity of greater than 1 μM) was noted at other pharmacologically important sites, including at the D₂.

An iterative process is then utilized with the results obtained in the each step being applied to subsequent modifications. The series of intermediates can be readily preparted as single enantiomers and both enantoimers are available. Towards this end the basic pharmacophore of the intermediates are modified in specific regions sequentially. After the preparation and biological characterization of the effects of each modification the remaining targets are modified to optimize their biological properties.

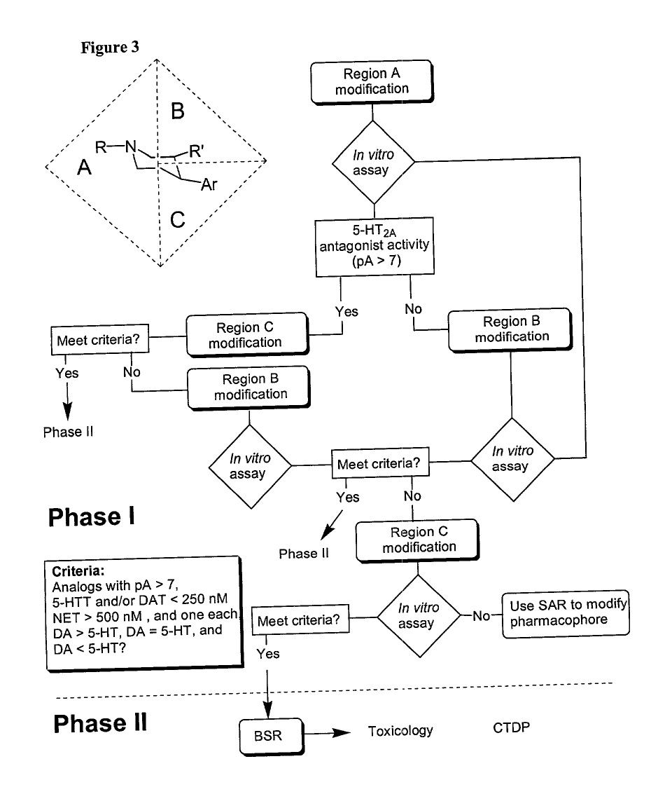

In one preferred embodiment, the decision making process will be employed as shown in FIG. 3. After the initial round of synthesis the lessons learned in this step are utilized to determine the best nitrogen substituent (Region A) to further study in the remaining modifications. In this manner, a concise rationally directed set of molecules for the treatment of cocaine abuse are prepared. As the development of effective medications for cocaine abuse is the goal at any time in which the prepared analogs exhibit the desired properties further synthetic manipulations will be suspended.

In another preferred embodiment, criteria for selection of desired analogs are as follows: 1) Agonist activity at the 5-HT_{2A} receptor of pA>8.2) Activity at DAT and or 5-HTT of greater than 250 nM (Kᵢ<250 nM) and NE activity of less than 500 nM. 3) One compound each exhibiting; DA>5-HT, DA 5-HT, and DA<5-HT.

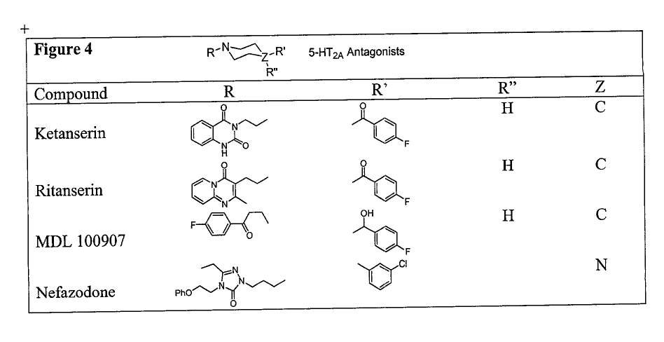

A wide variety of piperidino/piperazino ligands are known to exhibit high potency as 5-HT₂ antagonists. Some of the diverse structures are shown in FIG. 4. A number of structure activity studies have been reported with two basic regions, the nitrogen substituent (R in FIG. 4) and the 4-substituent (R′ and W″) being recognized as important for high 5-HT_{2A} antagonist activity. It is important to note that one of these 5-HT_{2A} antagonists, nefazodone which also exhibits inhibition of 5-HT uptake.

In one preferred embodiment, the reaction involving nitrogen substitution on the 5-HT_{2A} in FIG. (5) is then employed. Here (+)-2 is prepared and demethylated to afford (+)-5. Alkylation with the appropriately substituted alkyl halide will readily afford the 12 putative 5-HT_{2A} antagonists (6a-1).

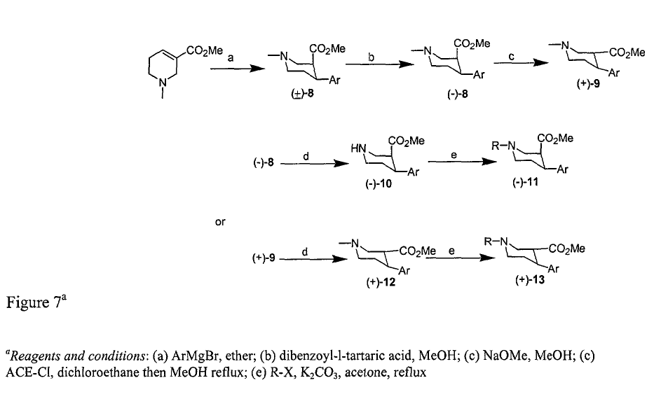

In one particular embodiment, the nitrogen substituent identified under the previous step that exhibits the optimum balance of 5-HT_{2A} and monoamine transporters activity are explored to optimize the effect of the methoxycarbonyl. substituent (Region B modification) (FIG. 7). No structure activity relationships exist for this substitution of the piperidino/piperizino ring of 5-HT_{2A} antagonists such as ketaserin. It is important to note however that 5-HT_{2A} antagonists of the lysergol family contain a 3,5-disubstituted piperidine ring system. In light of the rather dramatic differences in monoaminergic selectivity noted in previous studies as a result of the orientation of the 3-methoxycarbonyl group , the effects of this isomerization at the 5-HT_{2A} receptor are examined. The compounds prepared are all enantiomerically pure.

In another particular embodiment, the receptor-ligand interactions in the area encompassing the piperidine bridge will be mapped, yielding a SAR (Structure Activity Relationship). This will provide important additional information that will allow the rational design of ligands that meet the previously stated criteria.

In one particular embodiment, if decreases in the activity at the monoamine transporters are encountered it may be necessary to appropriately modify the aryl group of the piperidine to afford the desired monoamine potency selectivity. The largest effects on transporter activity/selectivity appear to be the result of the aryl substituent. (Foltin, R. W.; Fischman, M. W.; Lewin, F. R. Cardiovascular effects of cocaine in humans: laboratory studies. Drug Alc. Depend. 1995, 37, 193-210, Davies, H. M. L.; Kuhn, L. A.; Thomley, C.; Matasi, J. J.; Sexton, T.; Childers, S. R. Synthesis of 3β-aryl-8-azabicyclooctanes with high binding affinities and selectivities for the serotonin transporter site J. Med. Chem. 1996, 39, 2554-2558., Blough, B. E.; Abraham, P.; Lewin, A. H.; Boja, J. W.; Kuhar, M. J.; Carroll, F. I. Synthesis and transporter properties of 3β-(4′alkyl-, alkenyl-, and alkynylphenyl) nortropanes-2β-carboxylic acid methyl esters: serotonin transporter selective analogs. J. Med. Chem. 1996, 39, 4027-4035).

In one particular embodiment, using this established SAR, compounds with greater affinity for the DA and 5-HT transporters and with little or no NET activity will be prepared.

Assay

Measurements of the receptor (D₁, D₂, D₃, 5-HT_{1A}, 5-HT_{2A}, 5-HT_{2C} and 5-HT₃) binding affinity and functional assay is assayed, as well as binding and uptake at all three transporters. 5-HT_{2A} antagonist determinations are made using a rat aorta spiral tissue preparation. The test drug is incubated with the tissue sample and a concentration response curve is generated for 5-HT induced contraction of the tissue. Antagonist activity is obtained from the dose response curve before and after the addition of a single antagonist concentration. At least three different concentrations of the test drug are used. pA₂ values are determined from Schild plots using a statistical analysis. Inhibition of uptake at the three transporters DAT, 5-HT, and NET are measured and IC₅₀ values are calculated applying the GraphPAD Prism program to triplicate curves made up of 6 drug concentrations each. A tentative Kᵢ value is assigned to each compound based upon its IC₅₀ value and an assumption of a competitive mechanism. The Kᵢ values are estimated from two or three independent experiments for each measure.

The data obtained from the in vitro studies will be utilized to select compounds appropriate for further in vivo studies.

In a preferred embodiment, compounds exhibiting significant 5-HT_{2A} antagonist activity (pA>8) and inhibition of reuptake of 5-HT and/or DA (Ki<250 nM) are identified. Three compounds will be selected for preliminary in vivo studies encompassing a range of selectivities: one with 5-HT>DA, one with 5-HT DA, and one with DA>5-HT. Compounds that pass the above criteria are then tested in the BSR animal models for both their hedonic effects and the ability to antagonize cocaine's threshold lowering effects. Structural modifications are made to further improve the desired activity as they emerge from the animal studies.

Compounds of the Invention

In one aspect, the present invention provides a compound of formula (I)

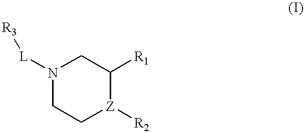

Where Z is NR₆, —C(R₄)(R₅)—, or —O—;

L is a (C1-C6)alkyl or (C1-C6)alkoxy, wherein any alkyl may be optionally substituted with 1, 2 or 3 substituents independently selected from halo, nitro, cyano, hydroxy, ketone, (C1-C6)alkoxy;

R₁ is —C(═O)ORₐ, cyano, (C1-C6)alkyl, (C1-C6)alkanoyl, (C2-C6)alkenyl, (C2-C6)alkynyl;

R₂ is (C6-C10)aryl, 5-10 membered heteroaryl, (C6-C10)aryl(C1-C6)alkyl, (C1-C6)alkyl(C6-C10)aryl, 5-10 membered heteroaryl(C1-C6)alkyl, (C6-C10)arylcarbonyl, biphenyl, or 5-10 membered heteroarylcarbonyl, wherein any aryl, biphenyl, or heteroaryl substituent may optionally be substituted on carbon with 1, 2, 3 substituents independently selected from the group consisting of halo, nitro, cyano, hydroxy, (C1-C6)alkyl, (C2-C6)alkenyl, (C2-C6)alknyl, (C1-C6)alkoxy, (C2-C6)acyloxy, trifluoromethyl;

R₃ is a (C6-C10)aryl, 5-10 membered heteroaryl, (C6-C10)aryl(C1-C6)alkyl, (C1-C6)alkyl(C6-C10)aryl, 5-10 membered heteroaryl(C1-C6)alkyl, (C6-C10)arylcarbonyl, biphenyl, heterocyclyls, or 5-10 membered heteroarylcarbonyl, wherein any aryl, biphenyl, or heteroaryl substituent may optionally be substituted on carbon with 1, 2, 3 substituents independently selected from the group consisting of halo, nitro, cyano, hydroxy, (C1-C6)alkyl, (C2-C6)alkenyl, (C2-C6)alknyl, (C1-C6)alkoxy, (C2-C6)acyloxy, trifluoromethyl;

R₄ and R₅ are independently hydrogen or (C1-C6)alkyl;

R₆ is a halogen, (C1-C6)alkyl, (C1-C6)alkanoyl, (C2-C6)alkenyl, (C2-C6)alkynyl, triflouromethyl, aryl(C1-C4)alkyl, heteroaryl(C1-C4)alkyl, aryl(C1-C4)alkanoyl, or heteroaryl(C1-C4)alkanoyl;

Rₐ is hydrogen, (C1-C4) alkyl, aryl, heteroaryl, aryl(C1-C4)alkyl, or heteroaryl(C1-C4)alkyl.

In a preferred embodiment, the compounds of the present invention are represented by the generalized structure (II)

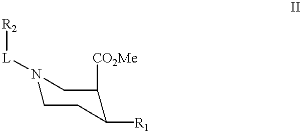

wherein

R₁ represents -alkylphenyl-, alkenylphenyl-, or alkynylphenyl or substituents.

L represents (C(R)₂)_{f} or M;

M is selected from the group consisting of

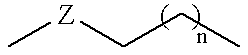

where n is 0, 1, or 2;

Z is C═O, CH₂, O

R represents independently for each occurrence H or alkyl;

f is 1, 2,or 3

R₂ is a (C6-C10)aryl, 5-10 membered heteroaryl, (C6-C10)aryl(C1-C6)alkyl, (C1-C6)alkyl(C6-C10)aryl, 5-10 membered heteroaryl(C1-C6)alkyl, (C6-C10)arylcarbonyl, biphenyl, heterocyclyls, or 5-10 membered heteroarylcarbonyl, wherein any aryl, biphenyl, or heteroaryl substituent may optionally be substituted on carbon with 1, 2, 3 substituents independently selected from the group consisting of halo, nitro, cyano, hydroxy, (C1-C6)alkyl, (C2-C6)alkenyl, (C2-C6)alknyl, (C1-C6)alkoxy, (C2-C6)acyloxy, trifluoromethyl.

In an even more preferred embodiment, the compounds of the present invention are represented by the generalized structure (III)

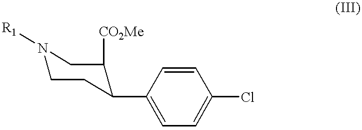

where R₁ is selected from the group

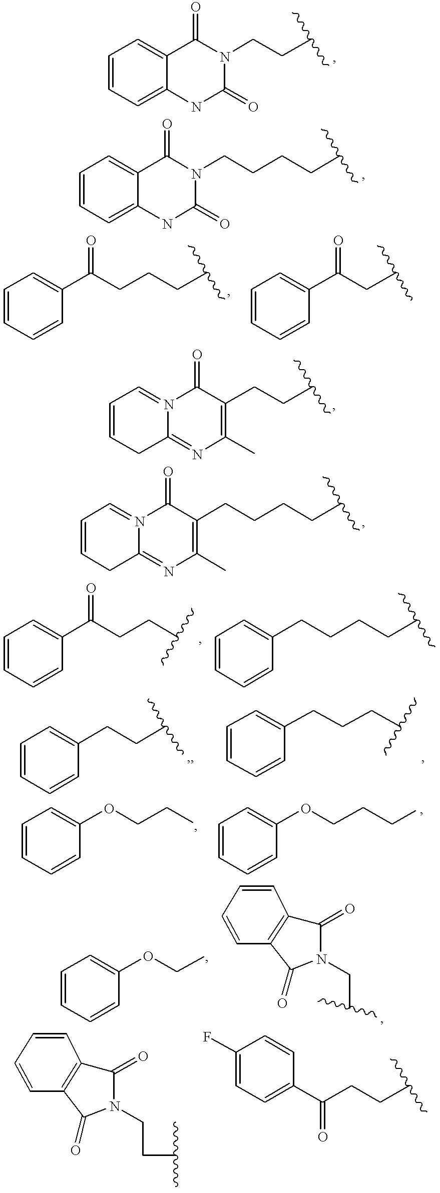

In one preferred embodiment, R₁ of compound III is a -napthyl substituent.

In a particular preferred embodiment, the compounds are selected from the group consisting of: methyl 4β-(4-Chlorophenyl)-1-(benzyl)piperidine-3β-carboxylate, methyl 4β-(4-Chlorophenyl)-1-(2′-phthalimidoethyl)piperidine-3β-carboxylate, methyl 4β-(4-Chlorophenyl)-1-(2′-phthalimidopropyl)piperidine-3β-carboxylate, methyl 4β-(4-Chlorophenyl)-1-[4-(4′-fluorophenyl)butan-4-one]piperidine-3β-carboxylate.

In another aspect, the present invention relates to a formulation, comprising a compound represented by generalized structure I, II or III, and a pharmaceutically acceptable excipient.

The present invention also provides a method of promoting 5-2HT_{2A} antagonistic activity in a mammal, comprising the step of administering to a mammal a therapeutically effective amount of a compound represented by generalized structure I, II, or III a formulation comprising a compound represented by generalized structure I, II, or III. In certain embodiments of this method, the mammal is a primate, equine, canine or feline. In certain embodiments of this method, the mammal is a human. In certain embodiments of this method, the compound or formulation is administered orally. In certain embodiments of this method, the compound or formulation is administered intravenously. In certain embodiments of this method, the compound or formulation is administered sublingually. In certain embodiments of this method, the compound or formulation is administered orally.

The present invention also provides a method of promoting inhibitory activity at the DAT, 5-HTT) and/or NET receptors and promoting 5-2HT_{2A} antagonistic activity in a mammal, comprising the step of administering to a mammal a therapeutically effective amount of a compound represented by generalized structure I, II, or III a formulation comprising a compound represented by generalized structure I, II, or III. In certain embodiments of this method, the mammal is a primate, equine, canine or feline. In certain embodiments of this method, the mammal is a human. In certain embodiments of this method, the compound or formulation is administered orally. In certain embodiments of this method, the compound or formulation is administered intravenously. In certain embodiments of this method, the compound or formulation is administered sublingually. In certain embodiments of this method, the compound or formulation is administered orally.

The present invention also provides a method of treating addiction to addictive substances, comprising administering to a patient having an addiction a therapeutically effective amount of a compound represented by generalized structure I, II, or III, or a formulation comprising a compound represented by generalized structure I, II, or III.

The present invention also provides a method of treating addiction to cocaine, comprising administering to a patient having an addiction to cocaine a therapeutically effective amount of a compound represented by generalized structure I, II, or III, or a formulation comprising a compound represented by generalized structure I, II, or III.

Pharmaceutical Compositions

In another aspect, the present invention provides pharmaceutically acceptable compositions which comprise a therapeutically-effective amount of one or more of the compounds described above, formulated together with one or more pharmaceutically acceptable carriers (additives) and/or diluents. While it is possible for a compound of the present invention to be administered alone, it is preferable to administer the compound as a pharmaceutical formulation (composition). As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra) Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrastemal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient. The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intravectally, for example, as a pessary, cream or foam.

The compounds according to the invention may be formulated for administration in any convenient way for use in human or veterinary medicine, by analogy with other pharmaceuticals.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers. Conjunctive therapy, thus includes sequential, simultaneous and separate administration of the active compound in a way that the therapeutical effects of the first administered one is not entirely disappeared when the subsequent is administered.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” O and B books, Corvallis, Oreg., U.S.A., 1977).

### EXAMPLES

The invention now being generally described, it will be more readily understood by reference to the following examples, which are included merely for purposes of illustration of certain aspects and embodiments of the present invention, and are not intended to limit the invention.

TABLE 1
Synthesis of representative N-substituted piperidines.
Reagents and conditions: i) RCl, K₂CO₃, MeCN reflux.

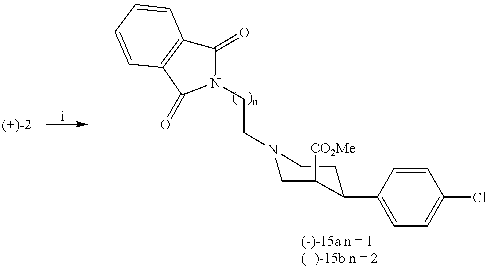

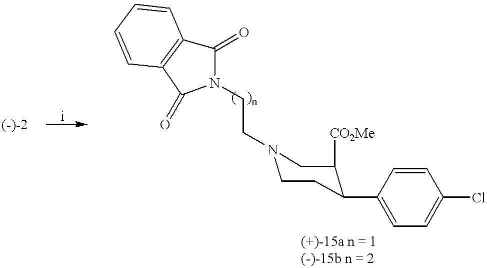

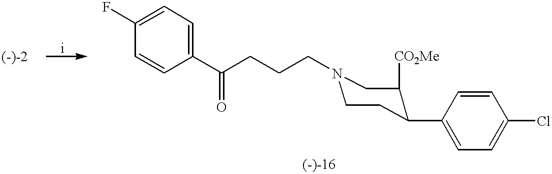

### Example 1

### (+)-Methyl 4β-(4-Chlorophenyl)-1-(benzyl)piperidine-3β-carboxylate, (+)-14

To a suspension of (+)-2 (5.9 g, 23 mmol), and K₂CO₃ (6.9 g, 50 mmol) in acetonitrile (100 mL) was added benzyl chloride (5.3 mL, 46 mmol). The resulting mixture was stirred at reflux for 3 h and then poured into satd. NaHCO₃ (100 mL) and diluted with ether (100 mL). The organic layer was separated and washed with water (100 mL), brine (100 mL) and dried (Na₂SO₄). Chromatography (hexanes/EtOAc, gradient) to afford (+) 14 (6.2 g, 78%) as a white solid: R_{f}=0.33 (hexanes/EtOAc, 9:1); [α]_{D}+41 (c 1.1, EtOH); mp=118-120° C.; ¹H NMR (d₆-DMSO) δ 1.78 (dd, 1H, J=2.0, 12 Hz), 2.19 (dt, 1H, J=2.8, 11 Hz), 2.30 (dd, 1H, J=4.2, 12 Hz), 2.46-2.59 (m, 1H), 2.80-2.87 (m, 1H), 3.00 (d, 1H, J=11 Hz), 3.07-3.10 (m, 1H), 3.36 (d, 1H, J=13 Hz), 3.41 (s, 3H), 3.63 (d, 1H, J=13 Hz), 7.24-7.34 (m, 9H); MS m/z (%) 343 (M⁺, 20), 312 (9), 284 (33), 252 (60), 91 (100).

### Example 2

### (−)-Methyl 4β-(4-Chlorophenyl)-1-(benzyl)piperidine-3β-carboxylate, (−)-14

Prepared from (−)-2 as described above to afford (−)-14 (87%) as a white solid: [α]_{D}-44.2 (c 0.98, EtOH); mp=117-119° C.

### Example 3

### −)-Methyl 4β-(4-Chlorophenyl)-1-(2′-phthalimidoethyl)piperidine-3β-carboxylate, (−)-15a

To a suspension of (+)-2 (450 mg, 1.8 mmol), and K₂CO₃ (690 mg, 5.0 mmol) in acetonitrile (100 mL) was added N-(2-bromoethyl)phthalimide (901 mg, 3.5 mmol). The resulting mixture was stirred at reflux for 18 h and then poured into satd. NaHCO₃ (50 mL) and diluted with ether (50 mL). The organic layer was separated and washed with water (50 mL), brine (50 mL) and dried (Na₂SO₄). Chromatography (hexanes/EtOAc, gradient) to afford (−)-15a (285 mg, 38%) as a white solid: R_{f}=0.45 (hexanes/EtOAc, 9:1); [α]_{D}-16.2 (c 1.05, CHCl₃); mp=179-180° C.; ¹H NMR (d₆-DMSO) δ 1.67 (d, 1H, J=6.6 Hz), 2.09 (m, 1H), 2.27-2.35 (m, 1H), 2.43-2.58 (m, 2H), 2.72-2.80 (m, 1H), 2.98-3.08 (m, 3H), 3.14 (d, 1H, J=11 Hz), 3.33 (s, 3H), 3.62-3.78 (m, 2H), 7.24-7.34 (m, 4H), 7.80-7.89 (m, 4H).

### Example 4

### (+)-Methyl 4β-(4-Chlorophenyl)-1-(2′-phthalimidopropyl)piperidine-3β-carboxylate, (+)-15b

Prepared from (+)-2 and N-(3-bromopropyl)phthalimide as described above to afford (+)-15b (80%) as a white solid: [α]_{D}+14.9 (c 0.98, CHCl₃); mp=139-140° C.

Example 5

### (+) -Methyl 4β-(4-Chlorophenyl)-1-(2′-phthalimidoethyl)piperidine-3β-carboxylate, (+)-15a

Prepared from (−)-2 and N-(2-bromoethyl)phthalimide as described above to afford (+)-15a (79%) as a white solid: [α]_{D}+15.0 (c 1.07, CHCl₃); mp=175-178° C.

### Example 6

### (−)-Methyl 4β-(4-Chlorophenyl)-1-(2′-phthalimidopropyl)piperidine-3β-carboxylate, (−)-15b

Prepared from (−)-2 and N-(3-bromopropyl)phthalimide as described above to afford (−)-15b (63%) as a white solid: [α]_{D}+16.2 (c 1.05, CHCl₃); mp=140-141° C.

### Example 7

### (−)-Methyl 4β-(4-Chlorophenyl)-1-[4-(4′-fluorophenyl)butan-4-one]piperidine-3β-carboxylate, (−)-16

Prepared from (−)-2 and 4-chloro-4′-fluorobutyrophenone as described above to afford (−)-16 (77%) as a white solid: [α]_{D}-127 (c 1.02, CHCl₃); mp=119-120° C.; ¹H NMR (d₆-DMSO) 1.72-1.84 (m, 3H), 2.09 (m, 1H), 2.26-2.44 (m, 4H), 2.79-2.86 (m, 1H), 2.91 (d, 1H, J=12 Hz), 3.05 (m, 2H), 3.11 (d, 1H, J=3.3 Hz), 3.20 (d, 1H, J=11 Hz), 7.32-7.39 (m, 4H), 8.01-8.06 (m, 4H).

Incorporation by Reference

All of the patentand publications cited herein are hereby incorporated by reference.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A compound represented by the generalized structure I: 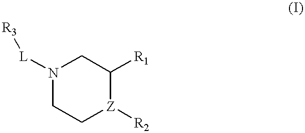
Where Z is NR₆, —C(R₄)(R₅)—, or —O—;
L is a (C1-C6)alkyl or (C1-C6)alkoxy, wherein any alkyl may be optionally substituted with 1, 2 or 3 substituents independently selected from halo, nitro, cyano, hydroxy, ketone, (C1-C6)alkoxy;
R₁ is —C(═O)ORₐ, cyano, (C1-C6)alkyl, (C1-C6)alkanoyl, (C2-C6)alkenyl, (C2-C6)alkynyl,;
R₂ is (C6-C10)aryl, 5-10 membered heteroaryl, (C6-C10)aryl(C1-C6)alkyl, (C1-C6)alkyl(C6-C10)aryl, 5-10 membered heteroaryl(C1-C6)alkyl, (C6-C10)arylcarbonyl, biphenyl, or 5-10 membered heteroarylcarbonyl, wherein any aryl, biphenyl, or heteroaryl substituent may optionally be substituted on carbon with 1, 2, 3 substituents independently selected from the group consisting of halo, nitro, cyano, hydroxy, (C1-C6)alkyl, (C2-C6)alkenyl, (C2-C6)alknyl, (C1-C6)alkoxy, (C2-C6)acyloxy, trifluoromethyl; and
R₃ is a (C6-C10)aryl, 5-10 membered heteroaryl, (C6-C10)aryl(C1-C6)alkyl, (C1-C6)alkyl(C6-C10)aryl, 5-10 membered heteroaryl(C1-C6)alkyl, (C6-C10)arylcarbonyl, biphenyl, or 5-10 membered heteroarylcarbonyl, wherein any aryl, biphenyl, or heteroaryl substituent may optionally be substituted on carbon with 1, 2, 3 substituents independently selected from the group consisting of halo, nitro, cyano, hydroxy, (C1-C6)alkyl, (C2-C6)alkenyl, (C2-C6)alknyl, (C1-C6)alkoxy, (C2-C6)acyloxy, trifluoromethyl;
R₄ and R₅ are independently hydrogen or (C1-C6)alkyl;
R₆ is a halogen, (C1-C6)alkyl, (C1-C6)alkanoyl, (C2-C6)alkenyl, (C2-C6)alkynyl, triflouromethyl, aryl(C1-C4)alkyl, heteroaryl(C1-C4)alkyl, aryl(C1-C4)alkanoyl, or heteroaryl(C1-C4)alkanoyl;
Rₐ is hydrogen, (C1-C4) alkyl, aryl, heteroaryl, aryl(C1-C4)alkyl, or heteroaryl(C1-C4)alkyl; and
The stereochemical configuration at any stereocenter of a compound represented by I may be R, S, or a mixture of these configurations,
and the pharmaceutically acceptable salts, esters, amides, and prodrugs thereof.

2. A formulation, comprising a compound claim 1; and a pharmaceutically acceptable excipient.

3. A compound represented by the generalized structure II: 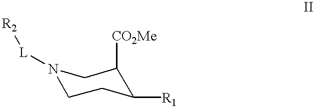
wherein
R₁ represents -alkylphenyl-, alkenylphenyl-, or alkynylphenyl or substituents.
L represents (C(R)₂)_{f} or M;
M is selected from the group consisting of 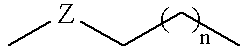
where n is 0, 1, or 2;
Z is C═O, CH₂, O
R represents independently for each occurrence H or alkyl;
f is 1, 2,or 3
R₂ is a (C6-C10)aryl, 5-10 membered heteroaryl, (C6-C10)aryl(C1-C6)alkyl, (C1-C6)alkyl(C6-C10)aryl, 5-10 membered heteroaryl(C1-C6)alkyl, (C6-C10)arylcarbonyl, biphenyl, heterocyclyls, or 5-10 membered heteroarylcarbonyl, wherein any aryl, biphenyl, or heteroaryl substituent may optionally be substituted on carbon with 1, 2, 3 substituents independently selected from the group consisting of halo, nitro, cyano, hydroxy, (C1-C6)alkyl, (C2-C6)alkenyl, (C2-C6)alknyl, (C1-C6)alkoxy, (C2-C6)acyloxy, trifluoromethyl; and
the stereochemical configuration at any stereocenter of a compound represented by II may be R, S, or a mixture of these configurations,
and the pharmaceutically acceptable salts, esters, amides, and prodrugs thereof.

4. A formulation, comprising a compound of claim 3; and a pharmaceutically acceptable excipient.

5. A compound, represented by the generalized structure III: 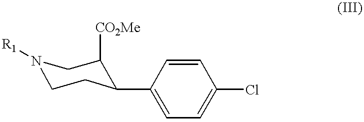
where R₁ is selected from the group consisting of 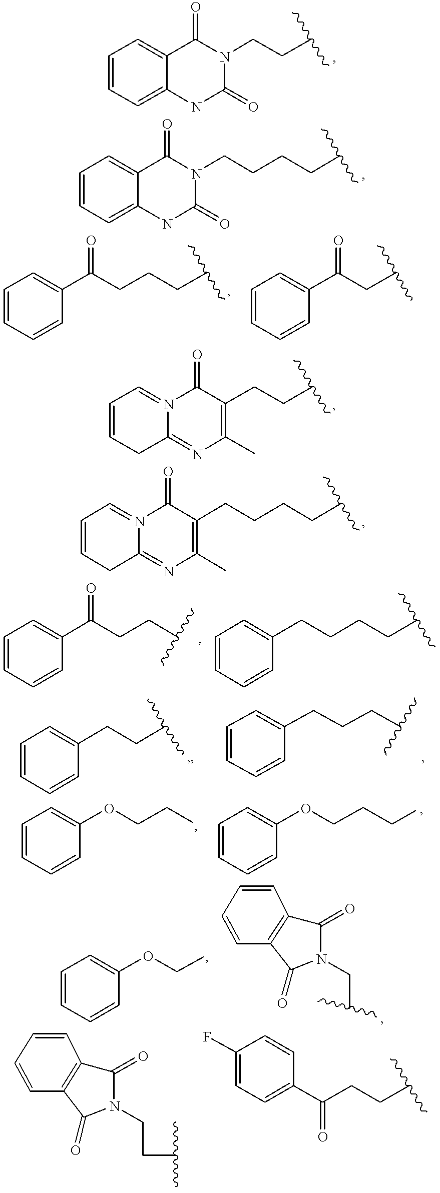
and the stereochemical configuration at any stereocenter of a compound represented by III may be R, S, or a mixture of these configurations,
and the pharmaceutically acceptable salts, esters, amides, and prodrugs thereof.

6. A formulation, comprising a compound of claim 5; and a pharmaceuticaly acceptable excipient.

7. The compounds methyl 4β-(4-Chlorophenyl)-1-(benzyl)piperidine-3β-carboxylate, methyl 4β-(4-Chlorophenyl)-1-(2′-phthalimidoethyl)piperidine-3β-carboxylate, methyl 4β-(4-Chlorophenyl)-1-(2′-phthalimidopropyl)piperidine-3β-carboxylate, methyl 4β-(4-Chlorophenyl)-1-[4-(4′-fluorophenyl)butan-4-one]piperidine-3β-carboxylate;
and the stereochemical configuration at any stereocenter of these compounds may be R, S or a mixture of these configurations,
and the pharmaceutically acceptable salts, esters, amides and prodrugs thereof.

8. A formulation, comprising a compound of claim 7; and a pharmaceutically acceptable excipient.

9. A method of promoting 5-2HT_{2A} antagonistic activity in a mammal, comprising the step of administering to a mammal a therapeutically effective amount of a compound of claim 1, 3, 5 or 7, or a formulation of claim 2, 4, 6 or 8.

10. The method of claim 9, wherein said mammal is a human.

11. The method of claim 9, wherein said compound or formulation is administered orally.

12. The method of claim 9, wherein said compound or formulation is administered intravenously.

13. The method of claim 9, wherein said compound or formulation is administered sublingually.

14. A method of promoting inhibitory activity at the DAT, 5-HTT and/or NET receptors and promoting 5-2HT_{2A} antagonistic activity in a mammal, comprising the step of administering to a mammal a therapeutically effective amount of a compound of claim 1, 3 5 or 7, or a formulation of claim 2, 4, 6 or 8.

15. The method of claim 14, wherein said mammal is a human.

16. The method of claim 14, wherein said compound or formulation is administered orally.

17. The method of claim 14, wherein said compound or formulation is administered intravenously.

18. The method of claim 14, wherein said compound or formulation is administered sublingually.

19. A method of treating of an addiction, comprising administering to a patient having the addiction a therapeutically effective amount of a compound of claim 1, 3, or 5, or a formulation of claim 2, 4, or 6.

20. The method of claim 19, where the addiction is to cocaine.
